(19) United States
(12) Patent Application Publication
(10) Pub. No.: US 2008/0132427 A1
(43) Pub. Date: Jun. 5, 2008

(54) CELL SYSTEMS AND METHODS FOR DETECTING PROLIFERATION ACITVITY

(76) Inventors: Yao Zhuang, Newbury Park, CA (US); Zheng Hu, Thousand Oaks, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/780,981

(22) Filed: Jul. 20, 2007

Related U.S. Application Data
(60) Provisional application No. 60/832,313, filed on Jul. 21, 2006.

(51) Int. Cl. C40B 40/02; C12N 5/06; G01N 33/567

(52) U.S. Cl. 506/14; 435/325; 435/354; 435/366; 435/7.21

(57) ABSTRACT

The invention provides proliferative response indicator cell having a vertebrate cell having a luciferase encoding nucleic acid and a heterologous proliferation factor receptor encoding nucleic acid, wherein each of the encoding nucleic acids are operationally linked to expression elements for co-expression of a luciferase polypeptide and a heterologous proliferation factor receptor. The invention also provides a method of determining a cell proliferative response to a proliferation factor. The method includes: (a) contacting a vertebrate cell expressing luciferase and a proliferation factor receptor with a proliferation factor for sufficient time for the proliferation factor to bind to the proliferation factor receptor; (b) culturing the contacted cell expressing luciferase for at least one generation, and (c) measuring the amount of light emission, wherein the luciferase expression is driven from a promoter non-responsive to the proliferation factor and the light emission directly correlates with proliferation factor-mediated cell proliferation. The proliferation factor can be a growth factor, a cytokine or a hormone or an agonist or antagonist thereof. The methods of the invention additionally include determining the effect of an inhibitor of the proliferation factor. Cells used in the method are contacted with a proliferation factor in the presence of a sample suspected of containing an inhibitor of the proliferation factor. The inhibitor can be neutralizing antibody, or binding fragment thereof, to the proliferation factor. The methods of the invention also are applicable as an indicator of cell health or viability. The invention further provides a diagnostic system. The diagnostic system includes a plurality of different vertebrate cell lines each encoding a luciferase gene and a different proliferation factor receptor, the luciferase gene being operationally linked to a promoter non-responsive to a proliferation factor bound by the proliferation factor receptor, wherein light emission from each of the different cell lines being characterized as directly correlating with proliferation factor-mediated cell proliferation.

This application is based on, and claims the benefit of, U.S. Provisional Application No. 60/832,313, filed Jul. 21, 2006, entitled CELL SYSTEMS AND METHODS FOR DETECTING PROLIFERATION ACTIVITY, and is incorporated herein by reference.

### BACKGROUND OF THE INVENTION

This invention relates generally to cellular proliferation factors and, more specifically to non-radioactive methods and cell systems for clinical assessment of proliferation factor activity.

With the advent of recombinant DNA technology, polypeptide-based therapeutics have become continually and increasingly commonplace in the repertoire of drugs available to medical practitioners for the treatment of a wide range of diseases from cancer to autoimmune diseases. Along with the scientific and technical advances that have occurred in the production of recombinant proteins, another reason for the success of protein therapeutics is their high specificity towards target molecules. The ability to employ biological molecules as pharmaceuticals in the treatment of diseases has significantly advanced medical care and quality of life over the past quarter of a century. As of the year 2005, there were more than one hundred and fifty approved polypeptide-based pharmaceuticals on the market and this number is expected to increase in the coming years.

Polypeptides known to exhibit various pharmacological actions in vivo are now capable of being produced in large amounts for various pharmaceutical applications. Stability and homogeneity of such a biopharmaceutical preparation is a particularly beneficial criterion for safe, consistent and efficacious treatments. However, despite the use of human polypeptides and safeguards in the preparation and approval process, the patient's own immune system can reduce or neutralize the effective concentration of an administered biopharmaceutical. Loss of biopharmaceutical functionality through host immune mechanisms can lead to loss of efficacy and risk of adverse side effects.

Many biopharmaceuticals approved for therapeutic treatment alter cell proliferative functions. For example, administration of factors such as erythropoietin, used in the treatment of anemia, and granulocyte colony-stimulating factor, used in the treatment of chemotherapy-induced neutropenia, serve to stimulate the growth and proliferation of target cells. For indications such as cancer, autoimmune disease and other pathologies characterized by uncontrolled cell growth, the biopharmaceutical generally targets molecules that inhibit cell proliferation. Thus, the ability to rapidly monitor the cell proliferative response of targeted cells during therapeutic treatment would provide significant clinical benefit in evaluating the effective levels of a biopharmaceutical regulating proliferation throughout the course of a therapeutic regime. Quick assessments as to the effective biopharmaceutical concentration as well as to the prognosis of the disease and efficacy of the therapeutic treatment could be made based on clinical measurements of proliferative responses. Such information could be used to adjust and alter the outcome of any particular therapeutic treatment.

Several methods exist for measuring cell proliferation. Such methods include the measurement of DNA synthesis or determining the viable number of cells within a sample of cultured cells. DNA synthesis measurements are based on incorporation of labeled DNA precursors into a cell's genetic material during DNA replication. The total amount of labeled precursor incorporated into the DNA in a population is correlated to the rate of cell division. One method of measuring DNA synthesis uses radioactive precursors such ³H-thymidine and, although this method can provide good sensitivity, the use of radioactivity in a clinical diagnostic setting is undesirable. Another DNA synthesis method uses 5-bromo-2′deoxyuridine BrdU, a non-radioactive DNA precursor. The BrdU incorporation method is often cumbersome and, therefore, is limited in assay throughput. One method relying on cell viability measures ATP content as an indicator of viability. This method can employ non-radioactive materials and is less cumbersome than the DNA synthesis methods. However, ATP content is an indirect measurement of cell proliferation and its use as a quantitative indicator for cell proliferation can be affected by numerous other cellular processes utilizing ATP as their main energy source. Therefore, use of this method as a reliable indicator of cell proliferative activities has not been fully established in clinical settings.

Thus, there exists a need for a rapid and efficient method for determining cellular proliferative activities as well as for determining effective concentrations of proliferative biopharmaceuticals. The present invention satisfies this need and provides related advantages as well.

### SUMMARY OF THE INVENTION

The invention provides proliferative response indicator cell having a vertebrate cell having a luciferase encoding nucleic acid and a heterologous proliferation factor receptor encoding nucleic acid, wherein each of the encoding nucleic acids are operationally linked to expression elements for co-expression of a luciferase polypeptide and a heterologous proliferation factor receptor. The invention also provides a method of determining a cell proliferative response to a proliferation factor. The method includes: (a) contacting a vertebrate cell expressing luciferase and a proliferation factor receptor with a proliferation factor for sufficient time for the proliferation factor to bind to the proliferation factor receptor; (b) culturing the contacted cell expressing luciferase for at least one generation, and (c) measuring the amount of light emission, wherein the luciferase expression is driven from a promoter non-responsive to the proliferation factor and the light emission directly correlates with proliferation factor-mediated cell proliferation. The proliferation factor can be a growth factor, a cytokine or a hormone or an agonist or antagonist thereof. The methods of the invention additionally include determining the effect of an inhibitor of the proliferation factor. Cells used in the method are contacted with a proliferation factor in the presence of a sample suspected of containing an inhibitor of the proliferation factor. The inhibitor can be neutralizing antibody, or binding fragment thereof, to the proliferation factor. The methods of the invention also are applicable as an indicator of cell health and/or viability. The invention further provides a diagnostic system. The diagnostic system includes a plurality of different vertebrate cell lines each encoding a luciferase gene and a different proliferation factor receptor, the luciferase gene being operationally linked to a promoter non-responsive to a proliferation factor bound by the proliferation factor receptor, wherein light emission from each of the different cell lines being characterized as directly correlating with proliferation factor-mediated cell proliferation.

### BRIEF DESCRIPTION OF THE DRAWINGS

FIG. 1 shows a dose response curve for 32Dcl23pPL-F11 cell proliferation stimulated with various concentrations of murine interleukin 3 (mIL-3).

FIG. 2 shows proliferation results measured by the luciferase assay compared to tritiated thymidine incorporation for cells stimulated with various concentrations of Thrombopoietin (TPO).

FIG. 3 shows proliferation results measured by the luciferase assay compared to ATP content determinations for cells stimulated with various concentrations of TPO.

FIG. 4 shows a direct correlation between cell proliferation and luciferase activity as assessed by growth curves where luciferase activity and viable, cell density were determined at different times following stimulated proliferation.

### DETAILED DESCRIPTION OF THE INVENTION

This invention is directed to a sensitive, non-radioactive method for efficiently detecting cell proliferation and cellular health state. The method uses luciferase expressing cell that synthesize luciferase in proportion to the amount of cellular growth or other proliferative characteristics of a cell. Light emission from the luciferase expressing cells of the invention is similarly proportional to the level of cellular growth and/or other cell proliferative characteristics. The method allows for shorter assay times compared to other methods such as tritiated thymidine incorporation and has a wide variety of applications including assessing cellular proliferation and/or viability as well as measuring the potency of proliferation factors that support the survival and/or growth of cells and tissues. The method also is applicable in clinical settings for assessing the presence of inhibitors and other neutralizing molecules of therapeutic compounds within a patient. The method is particularly useful for determining the presence or amount of neutralizing antibodies to biopharmaceuticals so as to rapidly obtain patient safety information before or during therapeutic treatments.

In one embodiment, the gene encoding Renilla luciferase was stably transfected into a thrombopoietin (TPO) receptor expressing cell line. Proliferation of this TPO receptor containing cell is stimulated in the presence of interleukin-3 (IL-3) or TPO. The luciferase encoding gene also was stably transfected into this cell line and constitutively expressed under the control of a CMV promoter. Renilla luciferase expression responded to the amount of IL-3 or TPO present in a dose dependent fashion. The luciferase expressing cells of this specific embodiment were used as assay readout to determine cell proliferation and health state as well as the effective concentration of proliferation factors within a sample.

In another embodiment, the use of luciferase expressing cells is used for the detection of neutralizing antibodies against one or more proliferation factors. An anti-proliferation factor antibody's capability to neutralize the activity of the proliferation factor, or therapeutic forms thereof, is a particularly useful diagnostic or prognostic tool for determining effective concentrations of a proliferation factor which allow timely adjustments of therapeutic treatment regimens.

As used herein, the term “luciferase” is intended to refer to the enzyme present in the cells of bioluminescent organisms that catalyzes the oxidation of luciferin to produce light. Luciferase and luciferin are general terms for an enzyme that catalyzes a light-producing reaction and its associated substrate. Therefore, the term “luciferase” includes enzymes derived from all species of organisms that catalyze the oxidation of a substrate to produce light. Exemplary species of luciferase included in the meaning of the term are the Renilla lucifersaes, which include enzymes found in, for example, Renilla reniformis (Sea Pansy soft coral) and Renilla mullerei (Gulf of Mexico soft coral); Firefly luciferase, which includes luciferases found within the family Lampyridae; Periphylla luciferase (Crown jellyfish), Noctiluca luciferase or Gonyaulax luciferase, both of which include any of various bioluminescent dinoflagellates of the genus Noctiluca and Gonyaulax, respectively; Omphalotus olearius (Jack O-Lantern mushroom) and luciferases found in any of various beetles of the family Elateridae (click beetles). Some organisms, such as the click beetles, have several different luciferase enzymes, each of which can produce different colors of emitted light from the same luciferin. The above exemplary luciferases as well as others well known in the art are all applicable in the cells, diagnostic systems and methods of the invention.

As used herein, the term “polypeptide indicator” is intended to mean an expressed polypeptide that can generate a measurable signal. Therefore, a polypeptide indicator refers to the product of a reporter gene. When used in reference to the measurement of a proliferative response, the term is intended to refer to an expressed polypeptide that can be used as determinant of a proliferation response. Exemplary polypeptide indicators of the invention include, for example, the various luciferases described above, Green fluorescent protein (GFP), variants of GFP, Yellow fluorescent protein (YFP), variants of YFP, Red fluorescent protein (RFP), other fluorescent proteins such as blue and cyan fluorescent proteins, β-galactosidase (β-gal), β-glucuronidase and β-lactamase. These and other polypeptide indicators are well known in the art and also are commercially available (see, for example, Clonetech, Mountain View, Calif.).

As used herein, the term “proliferation factor” is intended to mean a compound or polypeptide that alters cell proliferation or growth. Exemplary types of molecules included within the meaning of the term as it is used herein include growth factors, cytokines, hormones, chemokines and other biologic response modifiers. Alteration of cell proliferation or growth includes both stimulatory and inhibitory responses. Growth factors, cytokines, hormones and other biologic response modifiers are well known in the art to exhibit stimulatory and/or inhibitory cell proliferative functions. Specific examples of proliferation factors include the following growth factors, cytokines, hormones and biologic response modifiers.

Erythropoietin (EPO) is a glycoprotein hormone that is a cytokine for erythrocyte precursors in the bone marrow which regulates red blood cell production. A commercially available therapeutic form of this proliferation factor is EPOGEN® or Epoetin alfa, a mammalian cell expressed polypeptide of EPO (Amgen, Inc., Thousand Oaks, Calif.). A related form of this proliferation factor that is commercially available for therapeutic use is ARANESP® or darbepoetin alfa, which also is a mammalian cell expressed polypeptide variant of EPO having erythropoiesis stimulating activity (Amgen, Inc.).

Thrombopoietin (TPO) is a glycoprotein hormone produced by, for example, the liver and kidney that regulates the production of platelets by the bone marrow. TPO stimulates the proliferation and differentiation of megakaryocytes, which produce platelets.

Granulocyte colony-stimulating factor (G-CSF) is a glycoprotein, growth factor or cytokine produced by a number of different tissues that stimulates granulocyte production from bone marrow. G-CSF also stimulates the survival, proliferation, differentiation and/or function of neutrophil granulocyte progenitor cells and mature neutrophils. A commercially available therapeutic form of this proliferation factor is NEUPOGEN® or Filgrastim, which is an E. coli expressed human G-CSF (Amgen, Inc.). A related form of this proliferation factor that is commercially available for therapeutic use is NEULASTA® or pegfilgrastim, which is a covalent conjugate of recombinant methionyl human G-CSF and PEG (Amgen, Inc.).

Keratinocyte growth factor (KGF) is produced by cells of the connective tissue but controls epithelial proliferation and behavior. A commercially available therapeutic form of this proliferation factor is KEPIVANCE® or palifermin, which is a human recombinant KGF (Amgen, Inc.).

Interferon α is produced by leukocytes and stimulates macrophages and natural killer cells, for example. A commercially available therapeutic form of this proliferation factor is INFERGEN® or Interferon alfacon-1, which is an E. coli expressed recombinant polypeptide (Amgen, Inc.).

Stem Cell Factor (SCF) or c-kit ligand is a glycoprotein that plays an important role in hematopoiesis acting both as a positive and negative regulator. SCF often can act in synergy with other cytokines and also functions in mast cell development, gametogenesis and melanogenesis. A commercially available therapeutic form of this proliferation factor is STEMGEN® or Ancestim, which is a recombinant methionyl human stem cell factor (SCF) (Amgen, Inc.).

Soluble TNFα receptor, used as a biologic response modifier to inhibit TNFα function is a further example of a proliferation factor of the invention. A commercially available therapeutic form of this proliferation factor is ENBREL® or etanercept, a CHO expressed recombinant human soluble TNFα receptor polypeptide ((Amgen, Inc.).

An additional example of a biological response modifier includes soluble interleukin receptor antagonists. A commercially available therapeutic form of such a proliferation factor for the interleukin-1 receptor antagonist is KINERET® or anakinra, which is an E. coli expressed recombinant, nonglycosylated form of the human interleukin-1 receptor antagonist (IL-1Ra) (Amgen, Inc.).

The above exemplary proliferation factors are representative of the larger number of well known growth factors, cytokines, hormones, chemokines and other biological response modifiers included within the meaning of the term proliferation factor. Various other proliferation factors included within the meaning of the term are exemplified further below. Any of the proliferation factors exemplified herein as well others known in the art are applicable in the cells, systems and diagnostic methods of the invention.

As used herein, the term “proliferation factor-mediated” when used in reference to cell proliferation or growth is intended to mean the cellular proliferation stimulated or reduced by the referenced proliferation factor compared to cellular proliferation occurring in the absence of the referenced proliferation factor. Therefore, the term is intended to refer to cellular proliferation induced or inhibited by the referenced proliferation factor compared to baseline proliferation under the same or similar growth conditions. Similarly, the term “proliferation factor-mediated” when used in reference to cell cellular health state also is intended to mean the health state induced or inhibited by the referenced proliferation factor compared to the proliferative potential or health state occurring in the absence of the referenced proliferation factor. Accordingly, the term as it is used herein refers to a cell proliferation, growth or health state response directly or indirectly caused by a proliferation factor compared to a response in the absence of such factor.

As used herein, the term “promoter” when used in reference to expression of a nucleic acid is intended to mean the cis nucleic acid sequence controlling the rate of gene transcription located at or near the 5′ end of a gene that is recognized and bound by an polymerase of transcription. A “constitutive promoter” as it is used herein, is intended to refer to a promoter that actively transcribes at a relatively constant rate. An “inducible promoter” as it is used herein, is intended to refer to a promoter where its rate of transcription can be increased by a stimulus. A repressible promoter refers to a promoter where its rate of transcription can be decreased by a stimulus. Promoters, including constitutive, inducible and repressible promoters are well known in the art and can be found described in, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999), and Meyers, R. A., Molecular Biology and Biotechnology: A Comprehensive Desk Reference, VCH Publishers, New York, N.Y., (1995).

As used herein, the term “inhibitor” when used in reference to a proliferation factor is intended to mean a molecule that reduces the activity of a proliferation factor. Inhibitors can reduce the proliferation factor activity through a variety of different mechanisms including, for example, binding and prevention function or promoting and/or facilitating the degradation or clearance of the reference proliferation factor. A specific example of an inhibitor that can bind and prevent function of a proliferation factor is a neutralizing antibody. Generally, neutralizing antibodies binding at, or near the receptor binding site of a ligand and block the ability of the ligand to bind its receptor. Neutralizing antibodies also can bind elsewhere on the ligand and cause a conformation change that prevents or reduces the ligand's ability to bind receptor. Antibodies also can promote or facilitate the degradation and/or clearance of proliferation by binding and inducing cell mediated immune responses.

The invention provides a method of determining a cell proliferative response to a proliferation factor. The method includes: (a) contacting a vertebrate cell expressing luciferase and a proliferation factor receptor with a proliferation factor for sufficient time for said proliferation factor to bind to said proliferation factor receptor; (b) culturing said contacted cell expressing luciferase for at least one generation, and (c) measuring the amount of light emission, wherein said luciferase expression is driven from a promoter non-responsive to said proliferation factor and said light emission directly correlates with proliferation factor-mediated cell proliferation.

The methods of the invention utilize light emission from luciferase expressing cells as a quantitative or qualitative indicator of cell proliferation or other cellular attributes of proliferation. The methods are applicable to measuring cell proliferation or growth as well as cellular viability and/or health state. Light emission from luciferase expressing cells of the invention directly correlates with these and other cell proliferative characteristics.

The methods of the invention are applicable for determining proliferative responses for all cell-based systems including, for example, clinical diagnostic or prognostic cell-based determinations, cell culture systems, animal models and other in vitro, in situ and/or in vitro systems that can employ cell proliferative responses as an desired determinant. For example, the methods are applicable for measuring the levels of proliferation factors or inhibitors of proliferation factors as well as for determining the effective concentration of one or more proliferation factors in a sample because, for example, light emission from luciferase readouts directly correlates with cellular proliferative characteristics. Effective concentration is used herein to refer to the active fraction of polypeptides within a population of proliferation factors as measured by a functional assay or the in vivo specific activity of a proliferation factor.

The methods of the invention also are applicable for determining the amount of an inhibitor in a sample and/or the constituency of inhibitors in a sample by, for example, determining the level of reduction in activity of a proliferation factor in the presence or absence of the sample. Such determinations are particularly useful for determining the presence or levels of inhibitors such as neutralizing antibodies, proliferation factor receptor agonists or antagonists that can, for example, competitively or non-competitively inhibit the factor's actual activity. Other exemplary uses for the cells and methods of the invention include, for example, determining proliferation factor activity, mechanism and/or composition in a system. For example, the methods of the invention can be efficiently used to determine the number and types of factors in a system as well as to determine which proliferation factors should be included in a system to achieve optimal cell proliferative responses. Given the teachings and guidance provided herein, those skilled in the art will understand that there are a wide variety of applicable uses for the cells and methods of the invention.

The methods of the invention are exemplified herein with reference to determining a cell proliferative response to a proliferation factor. Given the teachings and guidance provided herein, those skilled in the art will understand that the methods also are applicable to the measurement of, for example, all cellular responses induced by proliferation factors that increase cell density, viability and/or health state. Similarly, given the teachings and guidance provided herein, those skilled in the art also will understand that the methods of the invention are equally applicable to determinations of both positive as well as negative effects of a proliferation factor on proliferative activity as well as to determinations of factor and/or inhibitor amounts and activities in a sample.

The methods of the invention include contacting a vertebrate cell expressing both luciferase, or other polypeptide indicator, and a proliferation factor receptor with a proliferation factor. As will be described further below, in some embodiments, the cells are recombinantly engineered to express both the polypeptide indicator and the proliferation factor receptor. In other embodiments, the cells are recombinantly engineered to express the polypeptide indicator and the endogenous proliferation factor receptor is utilized for proliferation factor-mediated cell growth.

A variety of polypeptides known in the art can be expressed in the cells of the invention as a non-radioactive indicator of one or more cell proliferative responses. The invention is exemplified herein with reference to luciferase expression and bioluminescent detection. However, any polypeptide that can be readily detected can alternatively be employed in the methods of the invention. Those polypeptides that are amenable to efficient or automated procedures are particularly useful for rapid determination of proliferation as a diagnostic or prognostic indicator. For example, alternatives to luciferase expression include reporter polypeptides such as β-galactosidase (β-gal), β-lactamase, Green Fluorescent Protein (GFP), other fluorescent proteins or enzymes such as alkaline phosphatase (AP). Expression and detection by, for example, bioluminescence, chemiluminescence, colorimetric, fluorescence, fluorescent activated flow cytometry (FACS) or appearance of enzymatic product or the disappearance or substrate of these and other well know reporter polypeptides are well known in the art.

Luciferase expression and luminescent detection of emitted photos is well known in the art and highly sensitive. As described previously, luciferase encoding nucleic acids from any bioluminescent species can be introduced and stably expressed in a cell for which proliferation or responsiveness to a proliferation factor is to be measured. Encoding nucleic acids for the different luciferase species are known in the art and their sequences can be found deposited in public databases including, for example, Genbank. Similarly, although the energy sources and substrates can vary between species, the oxidation of a luciferin to release light is substantially similar between the different species. For example, firefly luciferase utilizes ATP in its oxidation reaction whereas Renilla luciferase utilizes another energy source. However, both firefly and Renilla luciferase catalyze the oxidation of a luciferin to produce light. Renilla luciferase (soft coral), Photinus pyralis luciferase (firefly) or Photobacterium fischerii luciferase (bacterial) are particularly useful because they have been extensively characterized and reagents kits for detection are commercially available from several manufacturers.

Further, because some luciferases emit different wavelengths of light, the methods of the invention also are amendable to multiplexing different cell proliferation samples, detection of different proliferation factors and/or detection of different proliferation factor inhibitors based on color readout. For example, the proliferation response to a first proliferation factor can be assessed using a luciferase emitting a first color. In the same sample or in the same determination, the proliferation response to a second proliferation factor also can be assed using a luciferase emitting a second color. Similarly, the number of samples or detection measurements simultaneously measured in a sample or determination can include three, four or five or more and up to as many different emission wavelengths that are emitted by different luciferases. Accordingly, the invention provides for multiplex formats for detecting multiple different proliferative events in the same sample, in the same determination or both.

Similarly, the methods of the invention also can employ a multiplex format where different proliferative characteristics are determined simultaneously or determined simultaneously in the same sample by combining luciferase with another polypeptide indicator. For example, a proliferative response for a first proliferation factor can be assessed using luciferase expressing cells while a proliferative response for a second proliferation factor can be assessed using GFP or another well known calorimetric, fluorescent or enzymatic activity using cells expressing these types of polypeptide indicators. Generally, for example, the cells responsive to the first proliferation factor will be different from the cells responsive to the second proliferation factor. Using different cells for the different proliferation factors to be detected allows for efficient discrimination of the proliferation signals because each proliferation factor will be a different indicator signal that is associated with a different growth rate and/or proliferative response. Combining different types of polypeptide indicators will allow for multiplexing of 5, 10, 20 or more different samples rapidly in a format that is amenable to high through put automation.

By including different luciferases, colorimetric polypeptide indicators, fluorescent polypeptide indicators and/or enzymatic polypeptide indicators in various different combinations and/or permutations, including some or all combinations and permutations, very high levels of multiplex detection and/or sample through put can be achieved. For example, it is possible to achieve the simultaneous multiplex detection of 25, 30, 35, 40, 45 or 50 or more samples. Using such combinations of different polypeptide indicators it also is possible to achieve detection of 100, 200, 500, 1000 or more different samples in a single assay. The methods of the invention are applicable to multi-well plates and chip formats, which further allows for multiplex or high through put determinations of 10,000 to 1×10⁵ or more samples in a particular determination. All values above, below and in between the above exemplary sample numbers also can be determined using the methods of the invention. Methods and formats for automation of sample assays are well know in the art and can be used in connection with the methods of the invention for automated and/or multiplex formats to increase sample through put for rapidly determining proliferative characteristics in a large number of samples.

Cells used in the proliferation detection methods of the invention generally will be recombinantly engineered to stably express a luciferase or other polypeptide indicator encoding nucleic acid. One requirement for cell proliferation is a need to synthesize sufficient polypeptides and other macromolecules that can sustain the additional demands of a growing cell. This need results in the coupling of polypeptide biosynthesis to cell proliferative responses and can be utilized as an indicator of such cellular responses. Stable or consistent expression of a polypeptide indicator during a cell proliferative phase produces an accumulated amount of polypeptide indicator that is a consistent measurement of cell proliferation. Therefore, because the methods of the invention utilize stable expression of a polypeptide indicator the indicator signal provides a direct correlation between cell proliferation and total polypeptide indicator. A direct correlation can include, for example, a linear correlation.

Stable expression is characterized by, for example, consistent expression levels of the indicator polypeptide over multiple cell generations or doublings. Generally, stable expression includes, for example, consistent expression for greater than two weeks in culture. However, stable expression also can include consistent expression for about three weeks or more, particularly, four weeks or more, more particularly, five weeks or more, or for the life span of the cell expressing the polypeptide indicator. Consistent expression for time periods between or above these exemplary durations also is intended to refer to stable expression of a heterologously introduced nucleic acid. Stable expression also can include consistent expression for shorter periods of time, generally, greater than about three days. For example, consistent expression of about four days or more, about five days or more, about six days or more, about seven days or more also can be considered stable expression. Consistent expression of about 8, 9, 10, 11, 12, 13 or 14 days or more also can be considered stable expression of a luciferase or other indicator polypeptide of the invention. The actual generation time of a cell will vary depending on the doubling rate, growth conditions and other physiological considerations. The above time periods exemplify about 3 or more generations of cell division. The generation time for any particular cell or cell type can be readily determined by those skilled in the art.

Methods for recombinant engineering of cells and expression of heterologous of polypeptides are well known in the art. For example, most, if not all, of the more than 150 biopharmaceuticals approved for clinical indications have been recombinantly engineered and expressed as a heterologous polypeptide in either prokaryotic or mammalian cells. Such methods also can be found described in, for example, Sambrook et al., Molecular Cloning: A Laboratory Manual, Third Ed., Cold Spring Harbor Laboratory, New York (2001) and Ausubel et al., Current Protocols in Molecular Biology, John Wiley and Sons, Baltimore, Md. (1999).

Briefly, the luciferase or polypeptide indicator encoding nucleic acid can be, for example, obtained commercially, cloned, amplified by polymerase chain reaction (PCR) or other amplification method using known sequences in public databases, or chemically synthesized. As described further below, the luciferase or polypeptide indicator encoding nucleic acid is operationally linked to a promoter and/or other transcription and translation regulatory elements and inserted into a vector. Depending on the expression system chosen either the entire gene or cDNA can be used for expression. Alternatively, chimeric genes containing the luciferase encoding exons and one or more homologous or heterologous introns can be inserted for optimal polypeptide production in some eukaryotic expression systems.

Exemplary commercially available nucleic acids and expression vectors harboring nucleic acids encoding luciferase include, for example, pMAMneo-LUC (Clonetech, Mountain View, Calif.); pGL2-Basic, pGL2-Enancer, pGL2-Promoter and pGL2-Control (Promega, Madison, Wis.). Exemplary commercially available nucleic acids and expression vectors harboring nucleic acids encoding β-galactosidase include, for example, pβgal-Basic, βgal-Enhancer, βgal-Promoter, βgal-Control (Clonetech) and pSV-β-gal (Promega). Exemplary commercially available nucleic acids and expression vectors harboring nucleic acids encoding alkaline phosphatase include, for example, pSEAP-Basic, pSEAP-Enhancer, pSEAP-Promoter, pSEAP-Control (Clontech), pBC12/RSV/SEAP and pBC12/HIV/SEAP (Berger et al., Gene 66:1-10 (1988). Exemplary commercially available nucleic acids and expression vectors harboring nucleic acids encoding Green fluorescent protein include, for example, pEGFP, pEGFP1, pEGFP-N1,2,3 and pEGFP-C1,2,3 (Clonetech).

A wide variety of promoters and regulatory elements can be used to drive transcription of the luciferase encoding nucleic acid or other polypeptide indicator. As described further below, it is particularly useful to employ a promoter that is transcriptionally unlinked from the transcriptional effects of the proliferation factor activity that is to be measured. In this embodiment, luciferase transcription is unlinked from proliferation factor-mediated modulation of promoter activities and is, therefore, an accurate measurement of proliferation activity devoid of transcriptional effects.

Numerous promoters for eukaryotic expression are well known in the art and characterized with respect to transcriptional modulation by proliferation factors. Any of such promoters can be employed to drive transcription of the luciferase or other polypeptide indicator of the invention so long as they are transcriptionally non-responsive to the proliferation factor whose activity is to be measured. For example, in the specific instance where a proliferative response to bFGF is to be determined, a promoter that is transcriptionally regulated by bFGF is to be avoided if only proliferation activity is to be measured. Similarly, in a further specific instance where a proliferative response to PDGF is to be determined, avoidance of a promoter that is transcriptionally regulated by PDGF is preferably avoided.

Given the teachings and guidance provided herein, those skilled in the art will understand that inducible, cell-specific and other regulated promoters also are applicable and can be readily used in the methods of the invention so long as transcription induction is unlinked from the effects of the measured proliferation factor. For example, inducible promoters such as metallothionine and the like can be employed for inducible expression of luciferase or other polypeptide indicator. Similarly, promoters responsive to proliferation factors other than the one or ones whose proliferative effects are to be measured also can be employed for luciferase expression. In this specific embodiment, the induction of, for example, luciferase expression can occur before or during the proliferation measurements by adding the cognate inducer or factor for the selected promoter. Because transcription is non-responsive to the measured proliferation factor it will drive expression of the encoded polypeptide indicator at levels that correlate with the amount of cell proliferation. Various other inducible promoters are exemplified below with reference to their use in various known expression vectors or systems applicable to the methods of the invention.

Particularly useful promoters for luciferase or other polypeptide indicator expression include constitutive promoters that transcribe exogenous nucleic acids at moderate or strong levels. Use of constitutively active promoters ensures that luciferase or other indicator expression is unlinked from any effects of the proliferation factor to be measured. There are a wide variety of constitutive promoters well known in the art that can be operationally linked to luciferase or other polypeptide indicator encoding nucleic acid for consistent and reproducible expression during cell culture. As with the use of inducible promoters following an appropriate induction procedure, expression from such constitutively active promoters also will parallel the proliferative state of the cell since the encoded polypeptide will be synthesized, for example, at a steady state commensurate with the amount of other cellular polypeptide synthesis occurring given the energy state and growth requirements of the cell.

Specific examples of constitutive promoters useful in the cells employed in the methods of the invention include, for example, cytomegalovirus (CMV), thymidine kinase (tk), simian virus 40 (SV40) and phosphoglycerate kinase (PGK). The Example below describes the use of a CMV constitutive promoter transcribing a luciferase encoding nucleic acid where proliferation in response to TPO is measured. Various other constitutive promoters are exemplified below with reference to their use in various known expression vectors or systems applicable to the methods of the invention. Other constitutive promoters well known in the art also can be used to drive transcription of luciferase or another polypeptide indicator in a cell used in the methods of the invention.

Expression elements such as transcription and/or translation regulatory elements other than a promoter also can be operationally linked with the luciferase or polypeptide indicator encoding nucleic acid and included in the expression construct. Those skilled in the art of molecular biology will know what expression elements are useful and/or desirable to include in an expression construct given, for example, the expressing host cell, the selected promoter, media and desired level of transcription. Exemplary transcription expression elements other than a promoter that can be useful to operationally link to an encoding nucleic acid include, for example, enhancers, cell type-specific transcription factor binding sites, other cis regulatory elements, poly adenylation signal and/or a transcription termination sequence. Kirchhamer and Davidson, Development 122:333-346 (1996); Yuh and Davidson, Development 122:1069-1082 (1996); Davidson, E. H., Genomic Regulatory Systems: Development and Evolution, Academic, San Diego (2001) and in U.S. Patent Application 20040033601. Exemplary translation expression elements that can be useful to operationally link to an encoding nucleic acid include, for example, a ribosome binding site and/or other translational regulatory elements. Examples of other elements that can be included in an expression construct include introns and other gene structural elements as well as modifications to codon usage that substitute preferred codons of the expressing host species without changing or without substantially changing the amino acid sequence of the heterologous polypeptide. Such transcription and/or translation elements can be tailored to achieve a selected level of expression, regulation or other attributed desired for a particular application and/or for a particular cell type.

Selected promoters and other transcription and/or translation elements are operatively linked to a nucleic acid of interest such that the physical and functional relationship between the nucleic acid and the promoter and other elements allows transcription of the nucleic acid of polypeptide indicator. Suitable expression vectors are well-known in the art and include vectors capable of expressing nucleic acid operatively linked to a polypeptide indicator. Appropriate expression vectors include, for example, those that are replicable and that remain episomal or those which integrate into the host cell genome.

Suitable vectors for expression of heterologous nucleic acids in eukaryotic cells are well known to those skilled in the art (see, for example, Ausubel et al., supra). Vectors useful for expression in eukaryotic cells can include, for example, the promoters exemplified previously such as the SV40 early promoter, the cytomegalovirus (CMV) promoter, the phosphoglycerate kinase-1 promoter (PGK) promoter, thymidine kinase promoter (tk) as well as the adenovirus major late promoter, Moloney murine leukemia virus (MMLV) promoter, Rous sarcroma virus (RSV) promoter, elongation factor 1-alpha promoter (EF1α), the mouse mammary tumor virus (MMTV) steroid-inducible promoter, and the like. Vectors containing various promoters are commercially available (see, for example, Invitrogen, Carlsbad Calif.; Stratagene, San Diego Calif.; BD Biosciences Pharmingen, San Diego Calif.; Promega, Madison Wis.; Sigma-Aldrich, St. Louis Mo.; Novagen, Madison Wis.). An additional inducible mammalian expression system includes the RheoSwitch™ system using a chimeric bipartite nuclear receptor and synthetic inducer (New England BioLabs, Beverly Mass.). One skilled in the art will know or can readily determine an appropriate promoter or vector for expression in a particular host cell given the teachings and guidance provided herein.

Suitable vectors for delivering a nucleic acid encoding a polypeptide indicator of the invention to a vertebrate cell include, for example, viral vectors such as retroviral vectors, adenovirus, adeno-associated virus, lentivirus, herpesvirus, as well as non-viral vectors such as plasmid vectors (see, for example, U.S. Pat. No. 5,399,346, issued Mar. 21, 1995). Suitable viral vectors for introducing a nucleic acid into vertebrate cells are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)). Adenovirus-transferrin/polylysine-DNA (TfAdpl-DNA) vector complexes (Wagner et al., Proc. Natl. Acad. Sci., USA, 89:6099-6103 (1992); Curiel et al., Hum. Gene Ther., 3:147-154 (1992); Gao et al., Hum. Gene Ther., 4:14-24 (1993)) can be employed to transduce vertebrate cells with a nucleic acid of interest. Any of the plasmid expression vectors described herein may be employed in a TfAdpl-DNA complex.

Any of a variety of inducible promoters or enhancers can also be included in the vector for stable expression, under inducing conditions, of luciferase or other polypeptide indicator. Such inducible systems, include, for example, tetracycline inducible system (Gossen & Bizard, Proc. Natl. Acad. Sci. USA, 89:5547-5551 (1992); Gossen et al., Science, 268:1766-1769 (1995); Clontech, Palo Alto, Calif.); metalothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammory tumor virus (MMTV) induced by steroids such as glucocortocoid and estrogen (Lee et al., Nature, 294:228-232 (1981); and heat shock promoters inducible by temperature changes. One such inducible system utilizes a Gal4 fusion that is inducible by an antiprogestin such as mifepristone in a modified adenovirus vector (Burien et al., Proc. Natl. Acad. Sci. USA, 96:355-360 (1999). Another such inducible system utilizes the drug rapamycin to induce reconstitution of a transcriptional activator containing rapamycin binding domains of FKBP12 and FRAP in an adeno-associated virus vector (Ye et al., Science, 283:88-91 (1999)). It is understood that any combination of an inducible system can be combined in any suitable vector, including those disclosed herein. Such a regulatable inducible system can be particularly useful because the level of expression can be initiated or controlled by the amount of inducing agent administered.

The vector containing the luciferase or other polypeptide indicator encoding nucleic acid in an expressible form is introduced into the cells used in the proliferation detection methods of the invention. Cells are selected that stably express luciferase. Methods for stably introducing polypeptide encoding nucleic acids are well known in the art and include, for example, transfection, lipofection, viral particle transduction and electroporation. These and other methods of nucleic acid introduction are well known in the art and also can be found described in, for example, Sambrook et al., supra, and Ausubel et al., supra.

Cells chosen for use in the proliferation detection methods of the invention will contain a proliferation factor receptor. Generally, this receptor will correspond to the receptor that binds the proliferation factor whose activity is of interest. However, in certain embodiments, it is desirable to use the methods of the invention for detection of proliferative responsive irrespective of a cognate proliferation factor. For example, in some settings it is beneficial to determine the proliferation rate, proliferation activity or health state of a cell as a general cellular indicator or characteristic of a particular cell sample or cell type. In such embodiments, the proliferation factor receptor can be and endogenous receptor and the proliferation factor can be included as a normal constituent of the growth media. The receptor and/or the proliferation factor can be known or unknown to function in proliferation activity. In this specific embodiment, it is sufficient that the cell undergoes proliferative activity in culture and be modified to express luciferase or other polypeptide indicator as described herein.

In other embodiments, the cell used in the proliferation detection methods of the invention will be chosen to express, or be modified to express, one or more proliferation factor receptors. The proliferation factor receptor is chosen, for example, to correspond to the receptor that binds the proliferation factor for which activity is to be determined. Cells that endogenously express the receptor can be used without further modification beyond introduction of the luciferase encoding nucleic acids and expression elements. However, those skilled in the will understand that augmentation of receptor expression can be accomplished by introduction of further copies of the receptor encoding nucleic acid in an expressible form.

Cells that lack, or express undesirably low levels, of the proliferation factor receptor can be recombinantly engineered to stably express the receptor encoding nucleic acids. As with the luciferase encoding nucleic acids, the sequences for a large repertoire of proliferation factor receptors also are well known in the art and available in public databases such as Genbank. The promoters described above for expression of nucleic acids encoding luciferase or other polypeptide indicator can similarly be employed for expression of one or more proliferation factor receptors. The methods for vector construction, introduction and expression also are substantially the same as those exemplified above with reference to luciferase encoding nucleic acids and also can be found described in, for example, Sambrook et al., supra, and Ausubel et al., supra. Further, there are numerous successful reports of heterologous expression of membrane and soluble receptor expression. These and other methods well known in the art all are equally applicable for the introduction and expression of one or more heterologous nucleic acids encoding any chosen proliferation factor receptor.

Exemplified below in the Example is the construction and expression of a cell line stably expressing the proliferation factor receptor for TPO, termed 32Dcl23pRL-F11. These cells are particularly useful for determining cell proliferative responses to TPO. These cells also can be used to measure the effective concentration of TPO in a preparation of TPO or from a patient sample who has been treated with TPO. A further use for this TPO receptor expressing cell line is to measure the level of TPO inhibitors, such as neutralizing TPO antibodies, present in the serum of a patient treated with TPO.

Another exemplary cell line engineered to stably express a proliferation factor receptor is the cell line described in Wei et al., J. Immunol. Meth. 293:115-26 (2004), that has been modified to stably express the EPO receptor. This cell line is termed 32D-EPOR and is particularly useful for determining cell proliferative responses to EPO. As with the TPO receptor expressing cells describe above, these 32D-EPOR cells also can be used to measure the effective concentration of EPO in a preparation of EPO or from a patient sample who has been treated with EPO. 32D-EPOR cells can be further used for measuring the level of EPO inhibitors, such as neutralizing EPO antibodies, present in the serum of a patient treated with EPO.

Cells chosen for expression of luciferase, another polypeptide indicator and a proliferation factor receptor can be an vertebrate cell that that can proliferate or be made to proliferate. Cells exhibiting high proliferation rates are particularly useful because they allow for reduction in assay time compared to slower growing cell types. For example, hematopoietic cell lineages, fibroblasts, endothelial cells, liver cells, embryonic cell types, including embryonic kidney cells and transformed or immortal cell lines exhibit proliferation capabilities sufficient for use in a method of the invention. The above cell types are representative of the range of different cell types that can be employed in the methods of the invention. As those skilled in the art will understand given the teachings and guidance provided herein, a wide variety of other cell types are similarly applicable in the proliferation methods of the invention.

The cells used in the methods of detecting proliferative responses of the invention can be derived from any vertebrate species so long as it can be modified to express luciferase, or other polypeptide indicator, and expresses an endogenous proliferation factor receptor of interest, or also can be modified to co-express a proliferation factor of interest together with luciferase. Particularly useful cell types include established cell lines because they can be cultured for many generations and also are amenable to storage and other minor manipulations without substantially changing their proliferative characteristics. Alternatively, the methods of the invention also can utilize primary cells obtained from, for example, the patient to be diagnosed or who is currently under treatment, and cultured for sufficient time to enable genetic modification with luciferase encoding nucleic acids.

Methods for isolating cells are well known in the art. Alternatively, cells which have been previously characterized and isolated can be obtained from a commercial source, such as a tissue or cell bank (American Type Culture Collection, Rockville, Md.) and used directly for stable expression of heterologous luciferase or other polypeptide indicator and/or a proliferation factor receptor. Methods for the isolation of primary cells from a tissue source are well known in the art (see, for example, Freshney, Animal Cell Culture: A Practical Approach, 2nd ed., IRL Press at Oxford University Press, New York (1992). Maintenance of the cells prior to modification can be as a cell suspension, adherent cell culture or as organ culture. Conditions for the maintenance and culture of primary and clonal cells are well known in the art.

Mammalian cell types and cell lines are particularly useful in the methods of the invention because their growth properties have been well characterized and they are good in vitro systems indicative of in vivo cellular behavior. Useful mammalian cell types include, for example, human, mouse, rat, hamster and monkey.

Exemplified below in Example I is the use of 32D cells, which are mouse bone marrow derived cells (ATCC Cat. No. CRL-11346) of lymphoblast origin. Other specific types of cells applicable in the methods of the invention include, for example, AML-193, ELF-153, F-36P, GF-D8, GM/SO, HU-3, M-07e, MB-02, MHH-203, M-MOK, MUTZ-2, MUTZ-3, OCI/AML1, OCI/AML5, OCI/ML6, SKNO-1, TF-1, UCSD/AML1, UT-7, 293, NIH3T3, Hela, CHO, CV-1, PC-3, Jurkat and NFS60. These specific cell types are well known in the art and, any of which, can be employed in the methods of the invention. Numerous other cell types and cell lines well known in the art also can be similarly employed in the methods of the invention given the teachings and guidance provided herein.

The 32D cell line described in Example I has been modified to express the TPO receptor as described previously. Any of the above specific cell types or others well known in the art can be modified to express luciferase or to co-express luciferase and a proliferation factor receptor of interest. All of such specific cell types or others well known in the art are equally applicable in the proliferation assays of the invention. Therefore, the invention can utilize genetically modified 32D cells or any other genetically modified cell line as described herein including, for example, the representative cell types and cell lines described above.

Therefore, the invention provides an isolated cell stably co-expressing a heterologous luciferase or other polypeptide indicator and a heterologous proliferation factor receptor. Stable expression of such heterologous polypeptides can occur by, for example, introduction and selection of a cell line as described previously using methods well known in the art. Expression of luciferase or other polypeptide indicator nucleic acid can be accomplished using, for example, any of the constitutive or inducible promoters exemplified previously or known in the art. Expression of a heterologous proliferation factor receptor encoding nucleic acid also can be accomplished using, for example, any of the constitutive or inducible promoters exemplified previously or known in the art. Accordingly, the invention provides a cell line selected from, for example, AML-193, ELF-153, F-36P, GF-D8, GM/SO, HU-3, M-07e, MB-02, MHH-203, M-MOK, MUTZ-2, MUTZ-3, OCI/AML1, OCI/AML5, OCI/ML6, SKNO-1, TF-1, UCSD/AML1, UT-7, 293, NIH3T3, Hela, CHO, CV-1, PC-3, Jurkat or NFS60 and stably co-expressing a heterologous luciferase or other polypeptide indicator and a heterologous proliferation factor receptor. The proliferation factor receptor can include a receptor to any of the proliferation factors exemplified below or known in the art. For example, co-expression of a heterologous proliferation factor receptor can be selected from a receptor that binds to 5637 CM, bFGF, CNTF, EGF, EPO, FTL3L, G-CSF, GDNF, GM-CSF, HGF, IFN-α, IFN-β, IFN-γ, IGF-I, IGF-II, IL-1α, IL-2, IL-3, IL-4, IL-5, IL,6, IL-7, IL-8, IL-9, IL-10, IL-11, IL-12, IL-13, IL-15, IL-18, KGF, LIF, MCP-1, M-CSF, MIP-1α, NGF, OSM, PDGF, PIXY-321, PANTES, SCF, TGF-β, TNF-α, TNF-β, TPO, insulin, growth hormone, estrogen or progesterone.

Once a cell has been selected and modified as described above to exhibit desirable proliferative characteristics and/or to co-express luciferase, for example, and a proliferation factor receptor of interest, it can be employed in the proliferation detection methods of the invention. The methods include contacting such cells with a proliferation factor for which a proliferative response is to be determined. As described previously, in the specific embodiments where proliferation is to be measured without reference to a specific proliferation factor, it is sufficient to contact the cells with an appropriate growth medium. Determination of the level of light emission following culture will be indicative the growth rate or other proliferative response of the cell in the medium.

In other embodiments, it is desirable to determine a proliferative response with respect to an identified proliferation factor. Such a determination will yield not only the proliferation rate or level of the cells to the factor, but also will provide a measure of the effective concentration of the proliferation factor. In this specific embodiment, light emission following culture will be indicative of the growth rate or other proliferative response of the cell to the referenced proliferation factor. Light emission also will be indicative of the effective concentration of the proliferation factor in the sample when compared to light emission from the same or similar cells when contacted with a standard having a known amount of the proliferation factor.

Other proliferation responses that can be measured by the methods of the invention include, for example, cell density, viability and/or health state of a cell. Cellular growth is obligatorily coupled to the biosynthesis of polypeptides and other macromolecules. When rapidly proliferating, cells actively synthesize polypeptides, for example, that are used for cell growth and in other related processes. These polypeptides perform the requisite functions to sustain growth and viability during the increase in parental cell mass and for each daughter cell once cell division is completed. Therefore, during a rapidly proliferative state, polypeptide accumulation increases, for example, to supply the needed levels of the larger parental cell and to produce sufficient supplies for each daughter cell. The methods of the invention harness this coupling between polypeptide synthesis and proliferation to provide a rapid and sensitive measurement for proliferative responses. Responses related to cell growth include cell density, viability and health state. For example, increases in cell density is a consequence of cell proliferation. Higher polypeptide accumulation can occur due to, for example, the greater proliferation and/or the greater cell number relative to less dense cell populations. In comparison, a decrease in cell viability or in cell health state can result in lower levels of cell proliferation and a concomitant lower level of polypeptide synthesis. The converse is observed when cells are viable and/or healthy. Any of these proliferative responses as well as other responses known in the art can be quantitatively or qualitatively measured using the methods of the invention.

As described previously, use of a promoter that is transcriptionally non-responsive, or substantially non-responsive, to the proliferation factor will unlink expression from factor induced transcription. The luciferase or other polypeptide indicator product will be synthesized in direct proportion to the amount of cellular growth, cell density, viability or overall health state of the cell. Therefore, light emission, or other non-radioactive readout, from such cells also will directly correlate with cellular proliferation, density, viability or overall health state of the cell. The term a “direct correlation” is used herein to refer to production of a readout signal that is free of, or substantially free of proliferation factor-mediated transcriptional effects. Use of the promoters exemplified herein or others known in the art that are transcriptionally unmodulated by the proliferation factor whose activity is to be measured will result in luciferase or other polypeptide indicator signals that are free from proliferation factor-mediated transcriptional effects.

Cells used in the methods of the invention can be contacted with any one or more proliferation factors for which a proliferative response is desired. Numerous proliferation factors are well known in the art and, as described previously and include, for example, growth factors, cytokines, chemokines and biological response modifiers. The proliferative responses mediated by factors within any of these categories all are equally applicable for determination in the methods of the invention. Similarly, the effective concentration of factors within any of these categories also are all equally applicable for determination using the methods of the invention. Proliferative responses and effective concentrations of factors within other categories known in the art also can be measured using the methods of the invention.

Specific examples of proliferative factors applicable in the methods of the invention include those factors exemplified previously as well as 5637 CM, bFGF, CNTF, EGF, EPO, FTL3L, G-CSF, GDNF, GM-CSF, HGF, IFN-α, IFN-β, IFN-γ, IGF-I, IGF-II, IL-1α, IL-2, IL-3, IL-4, IL-5, IL,6, IL-7, IL-8, IL-9, IL-10, IL-11, IL-12, IL-13, IL-15, IL-18, KGF, LIF, MCP-1, M-CSF, MIP-1α, NGF, OSM, PDGF, PIXY-321, PANTES, SCF, TGF-β, TNF-α, TNF-β, TPO, insulin, growth hormone, estrogen or progesterone. Other exemplary factors include any of the cell growth modulators referenced previously that are approved for therapeutic indications or undergoing clinical trials for therapeutic approval. Other proliferative factors also include, for example, agonist or antagonists of the proliferation factor receptor. Proliferative factor characteristics and activities representative of those listed above as well as others are well known in the art.

The proliferation factor for which a response is to be determined will generally be obtained from a sample known to contain, or suspected to contain, the proliferation factor of interest. For example, and as described previously, the methods of the invention are particularly useful for determining the effective concentration of a proliferation factor in a sample. The method includes contacting cells of the invention with a sample and determining the amount of indicator signal such as light emission following culturing for at least one generation. With exemplary reference to a luciferase polypeptide indicator, the amount of light will be directly proportional to the amount of active proliferation factor in the sample. A cell of the invention also can be contacted with a control having a known amount of the proliferation factor of interest. The amount of light emission from the cells contacted with a test sample relative to the amount emitted in the presence of the control provides a relative comparison of the amount of active proliferation factor present in the sample. Signal-to-noise, or sensitivity, can be determined by contacting a cell of the invention with a control known to be deficient of the proliferation factor interest. As exemplified in the Examples, the methods of the invention are highly sensitivity, resulting in sensitivities of greater than 5, 10, 50, 100 or 500-fold or more compared to, for example, DNA synthesis methods.

Other sources of proliferation factors for which determination of a proliferative response include, for example, commercial manufacturers, recombinant methods using known encoding nucleic acid sequences deposited within public databases such as Genbank, chemical synthesis and/or biochemical purification. The nucleotide and amino acid sequences of a large variety of proliferation factors are well known in the art. Similarly, the activities and characteristics also are well known in the art. Therefore, the above methods as well as other procedures well known in the art all are equally applicable for obtaining a source of proliferation factor for use in the methods of the invention.

A cell used in the methods of the invention is contacted with a proliferation factor or a sample suspected of containing a proliferation factor as described previously. Contacting is for an amount of time sufficient for the proliferation factor to bind its cognate proliferation factor receptor and induce a proliferative response. Generally, such times include, for example, periods from about 5-120 minutes, more particularly between about 10-90 minutes, particularly between about 15-60 minutes and can be between about 20-30 minutes. Time periods shorter, longer or in between these exemplary ranges also can be sufficient for a proliferation factor to bind its receptor and induce a proliferative response. For example, the time periods described in Example I for incubation with TPO include an incubation of greater than 120 minutes, corresponding to about 8-12 hours or more. Therefore, an amount of time sufficient for the proliferation factor to bind its cognate proliferation factor receptor and induce a proliferative response also includes, for example, periods of about 15, 18, 24, 36, 48, 60 or 72 hours or more. Those skilled in the art will know what incubation time of cells with proliferation factors is sufficient for binding and induction of a proliferative response.

Following the contacting step, the cells are cultured for a sufficient time to allow changes in proliferative activities to be measured. In certain embodiments, this time period will be at least for one generation period or a doubling time. However, the cells can be cultured for more than one generation time including, for example, 2, 3, 4, 5, 6, 7, 8, 9 or 10 or more generations. Alternatively, in certain embodiments, the proliferative responses can be measured under continuous growth conditions where media, nutrients and proliferation factor or factors are continuously infused and spent media is removed. Continuous culture conditions are particularly useful for determining proliferation rates stimulated by a proliferation factor of interest.

Signal from the expressed luciferase is determined by measuring photon emission in the presence of luciferin substrate using, for example, a luminometer or scintillation counter. Luciferin substrate, luciferase reaction buffers and other reagents for detecting luciferase catalytic activity are well known in the art and are commercially available. Preparation and detection of emitted light from stably transfected luciferase expressing cells is exemplified below in Example I. Other luciferase detection procedures known in the art are equally applicable for use in the methods of the invention and can be found described in, for example, Ausubel et al., supra, Supp. 37.

Similarly, when other luminescent polypeptide indicators, calorimetric or fluorescent polypeptide indicators are used in substitution of luciferase, or when used in addition with luciferase, the presence of the indicator polypeptide is determined by measuring the appearance of a light, colorimetric or fluorescent signal. For example, β-galactosidase, β-glucoronidase (GUS) and β-lactamase are amenable to colorimetric, fluorescent or chemiluminescence detection. Alkaline phosphatase is amenable to colorimetric, bioluminescence or chemiluminescence detection. Green fluorescent protein is amenable to fluorescent detection. Such methods are well known in the art and can be found described in, for example, Sambrook et al., supra, and Ausubel et al., supra, as well as in Harlow and Lane., Antibodies: A Laboratory Manual, Cold Spring Harbor Laboratory Press (1989). Further, colorimetric and fluorescent substrates, reaction buffers and other reagents for detection also are commercially available and can be obtained in, for example, kits for ease of use in connection with a wide variety of applications. Methods for fluorescent activated flow cytometry can be found described in, for example, Shapiro, H. M., Practical Flow Cytometry, 3rd Ed., Wiley-Liss, NY, (1995).

The invention also provides a method of determining a cell proliferative response to a proliferation factor that measures the effect of an inhibitor of the proliferation factor. The method includes the step of contacting the vertebrate cell expressing luciferase and a proliferation factor receptor with a proliferation factor in the presence of a sample suspected of containing an inhibitor of the proliferation factor, wherein a decrease of cell proliferation in the presence of the sample indicates that the sample includes an inhibitor of the proliferation factor.

As described previously, the methods of the invention are particularly useful in for determining a response to a proliferation factor from a sample known to contain, or suspected to contain, a proliferation factor of interest. One specific example of such as sample is a blood or fluid sample from a patient undergoing therapy with a proliferation factor. In certain circumstances it can be desirable to diagnostically determine the amount or level of an inhibitor of the proliferation factor present in the individual. Such a diagnosis can be performed by, for example, determining the effective concentration of the proliferation factor in the sample. A proliferative response less than that obtained relative to a control having substantially the same amount of active proliferation factor compared to the sample indicates the presence of an inhibitor in the sample.

For formulation of the control having a known and comparative amount of proliferation factor relative to the sample, those skilled in the art will understand that the amount of both active and inactive proliferation factor molecules present in a sample can be determined by a variety of well known methods. Such methods can include, for example, immunoaffinity determinations using proliferation factor binding polypeptides. Additionally, given the well known field of pharmacokinetics, those skilled in the art also can predict the amount of proliferation factor present in a sample from, for example, the amount administered, the duration of administration and/or the clearance rate of the proliferation. Once the amount of proliferation factor in a sample is determined or predicted, a control can be formulated containing this amount and used in the methods of the invention. Signals obtained that are less than those produced by the control directly correlate with the level of inhibitor present in the patient sample. Therefore, the amount of light produced in the presence of an inhibitor of the proliferation factor will be directly proportional to the amount of active factor.

The above specific embodiment of the invention is particularly useful for diagnosing the presence of inhibitors produced by a patient's own immune system against the therapeutic proliferation factor and/or endogenous counterpart of the therapeutic protein, which results in serious consequence to the patient (Casadevall et al., New England Journal of Medicine 346:469-75 (2002). Such natural inhibitors include, for example, neutralizing antibodies to the proliferation factor and/or to the proliferation factor receptor as well as antagonists to the receptor. The neutralizing antibodies or other inhibitors of the proliferation factor can function to reduce the activity of the factor by a variety of mechanisms including, for example, binding and clearance, competitive and/or non-competitive inhibition. Such molecules and methods of action are well known in the immunological and therapeutic arts. Given the teachings and guidance provided herein, the methods of the invention are equally applicable to their detection and/or determination of their inhibitory activity.

The invention also provides a diagnostic system. The system includes a plurality of different vertebrate cell lines each encoding a luciferase gene and a different proliferation factor receptor, the luciferase gene being operationally linked to a promoter non-responsive to a proliferation factor bound by the proliferation factor receptor, wherein light emission from each of the different cell lines being characterized as directly correlating with proliferation factor-mediated cell proliferation.

The cells and methods described above can be accumulated into a diagnostic system for determination of a plurality of different proliferative responses to a variety of different proliferative factors. As used herein, the term “plurality” is intended to mean a population of two or more. The invention includes small pluralities containing, for example, 2, 3, 4, 5, 6, 7, 8, 9 or 10 or more different modified cells and/or a comparable number of different proliferation factors. The invention also includes large pluralities containing, for example, 20, 30, 40, 50, 100, 200 or 500 or more different modified cells and/or a comparable number of different proliferation factors. Pluralities of cells and/or proliferation factor receptors described above or in between these numbers also are included in the diagnostic systems of the invention.

The diagnostic system of the invention can include, for example, a cell panel or bank, each expressing luciferase and a proliferation factor receptor of interest. Such a cell panel of cells can be used, for example, as a broad based diagnostic or prognostic tool for determining proliferative responses to endogenous proliferation factors, exogenous proliferation factors or both endogenous and exogenous proliferation factors. Therefore, the system of the invention is applicable to the determination of one or more proliferative responses to some or all proliferation factors in an individual.

The diagnostic system of the invention is neither restricted by the large number of different proliferation factors present in an individual nor by the variety of different activities stimulated or inhibited by these factors. A diagnostic system can be generated as described above for the cells used in the methods of the invention. Those skilled in the art can select one or more promoters as described above and recombinantly engineer each cell to co-express luciferase and a different proliferation factor receptor of interest, including all proliferation factor receptors known or available. Therefore, a diagnostic system of the invention can include a few, some or all of the cells exemplified above or cell expression all known or available proliferation factors to each express a polypeptide indicator and a different proliferation factor receptor.

Included in the diagnostic system can be one or more ancillary reagents useful for culturing, stimulating one or more proliferative responses, inhibiting one or more proliferative responses, detection of luciferase signal, detection of another polypeptide indicator, buffers, substrates and/or any combination thereof. Additionally, a diagnostic system of the invention also can include, for example, one or more proliferation factors, one or more control factors and/or control cells. Instructions for using any or all of these ancillary reagents also can be included in a diagnostic system of the invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Cell Proliferation Determination by Luciferase Expression

This Example shows that cell proliferation and/or cell viability can be assessed by luciferase activities constitutively expressed in stably transfected cells.

The cell line 32Dcl23 is a stably transfected 32D cell line that expresses human mpl gene encoding the receptor for Thrombopoietin (TPO). These 32D clone 23 cells are dependent on recombinant murine interleukin-3 (IL-3) for routine culturing and growth and respond to both TPO and mIL-3 with cell proliferation. Responses to cytokine induced proliferation was determined by light emission from constitutively expressed luciferase and compared to tritiated thymidine and ATP content proliferation assays.

The TPO receptor-expressing stable cell line constitutively expressing Renilla luciferase, termed 32Dcl23pRL-F11, was generated by cotransfection of pRL-CMV (Promega, Madison, Wis.) and pBK-CMV (Stratagene, San Diego, Calif.) plasmids into 32Dcl23 cells. pRL-CMV is a plasmid which contains wild type Renilla luciferase gene constitutively expressed under the CMV promoter. pBK-CMV is an empty expression vector which provided the G418 resistance as a selection marker. The proliferation of 32Dcl23pRL-F11 stimulated by mIL-3 and TPO was measured via light emission to determine luciferase activities in the cells. Light emitted from the oxidization reaction of coelenterazine to coelenteramide in presence of Renilla luciferase was recorded in the luminometers.

To perform the luciferase proliferation assays, 32Dcl23pRL-F11 cells were maintained in growth media (MEM, 10% Fetal Clone II, 1% penicillin/streptomycin) in presence of 1 ng/mL of mIL-3 and 600 μg/mL Geneticin. The luciferase assays were performed as follows. On day 1, the cells were washed three times with assay media and the cell density was determined by cell counting. Cells were resuspended in assay media to a density of 1×10⁵ cells/ml and plated in 50 μl aliquots to each well of a 96-well plate. Cytokine reagent, 50 μl consisting of either mIL-3 or TPO prepared growth medium, was added to each well and the cells were incubated at 36° C. (±2° C.), 10% CO₂ (±2%) and 90% humidity (±10%) overnight.

On day 2, luciferase activities were measured using the Dual-Glo Luciferase Assay System according to manufacture's recommendations (Promega). Briefly, 100 μl of Dual-Glo Luciferase buffer (Promega, Madison, Wis.) without firefly luciferase substrate was added to each well of the plated cells and incubated at ambient temperature for 20±5 minutes. The luciferase reactions were performed by adding 100 μl of Dual-Glo Stop and Glo reagent prepared according to manufacture's instructions (Promega) to each well and incubated in dark at ambient temperature for 30±15 minutes. Renilla luciferase activities were measured with a luminescence reader for 1 sec per well.

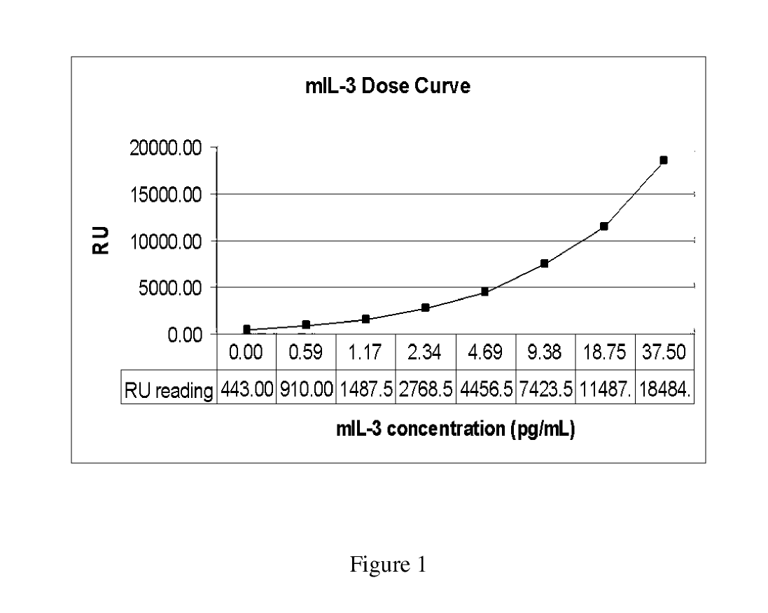

FIG. 1 shows a dose response curve for 32Dc123pPL-F11 cell proliferation stimulated with mIL-3. Cells free of mIL-3 were incubated with 0, 0.59, 1.17, 2.34, 4.69, 9.37, 18.75 and 37.5 pg/mL of mIL-3 overnight. Proliferation was determined by the luciferase assay described above. As shown by the curve in FIG. 1, the cells responded by proliferation in a dose dependent manner when treated with the various mIL-3 concentrations. In particular, at 5 pg/mL of mIL-3, the signal to background ratio was about 10-fold. A similar dose dependent proliferation response also was observed using this luciferase assay when cells were stimulated with TPO.

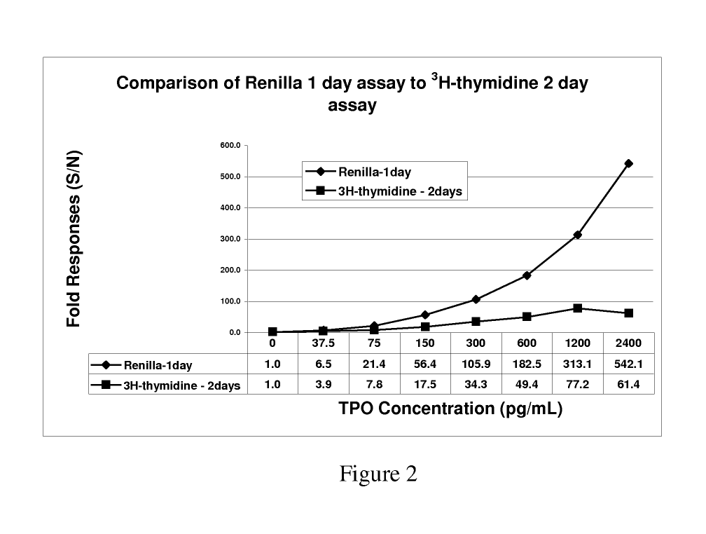

FIG. 2 shows TPO stimulate proliferation results measured by the luciferase assay compared to tritiated thymidine incorporation. Cells free of mIL-3 were incubated with 0, 37.5, 75, 150, 300, 600, 1200 and 2400 pg/mL of TPO overnight for the luciferase proliferation assay or two days for the ³H-thymidine uptake assay.

The tritiated thymidine proliferation assays were performed as follows. Briefly, on day 1, cells were washed three times with assay media and the cell density was determined by cell counting. Cells were resuspended in assay media to a density of 8×10⁵ cells/ml and incubated at 36° C. (±2° C.), 10% CO₂ (±2%) and 90% humidity (±10%) overnight.

On day 2, the cells were washed once with assay media and cell density again determined by cell counting. Cells were resuspended in assay media to a density of 4×10⁵ cells/ml and plated in 100 μl aliquots to each well of 96-well plate. Cytokine reagent, 100 μl consisting of TPO formulated in buffer or growth medium, was added to each well and the cells were incubated overnight at 36° C. (±2° C.), 10% CO₂ (±2%) and 90% humidity (±10%).

On day 3, 40 μCi/mL of ³H-Thymidine was prepared in assay media and 50 μl was added to each well of the 96 well microtiter plate. The cells were further incubated at 36° C. (±2° C.), 10% CO₂ (±2%) and 90% humidity (±10%) for 4±0.5 hours. Following incubation, cells were harvested to a Unifilter plate through Packard FilterMate Harvester and the plate was dried. Micro scintillation reagent (35 μl) was added to each well and the plate was sealed with plate sealer. Plates were read on a TopCount Reader for 1 minute per well.

As shown in FIG. 2, the response measured by the luciferase proliferation assay was higher compared to the ³H-thymidine method at all the concentrations tested. In particular, starting at a TPO concentration of 75 pg/mL, a significantly higher proliferation response was observed for the luciferase assay compared to ³H-thymidine incorporation. These results indicate that proliferation or growth measured by luciferase activity provide more robust readouts and sensitivities compared to ³H-thymidine incorporation assays.

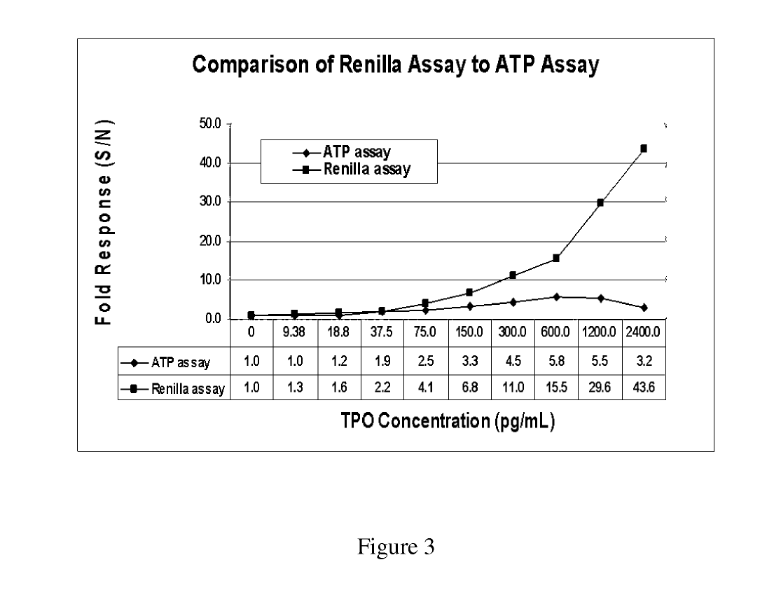

FIG. 3 shows TPO stimulated proliferation results measured by the luciferase assay compared to ATP content as a measure of proliferation. Cells free of mIL-3 were incubated with 0, 37.5, 75, 150, 300, 600, 1200 and 2400 pg/ml of TPO overnight for both the luciferase proliferation and ATP assays.

Cellular ATP content was measured as follows. Briefly, on day 1, cells were washed three times with assay media and the cell density was determined by cell counting. Cells were resuspended in assay media to a density of 1×10⁵ cells/ml ml and plated in 50 μl aliquots to each well of 96-well plate. Cytokine reagent, 50 μl consisting of TPO formulated in buffer or growth medium, was added to each well and the cells were incubated at 36° C. (±2° C.), 10% CO₂ (±2%) and 90% humidity (±10%) overnight.

On day 2, ATP content was measured using the Cell Titer-Glo System according to the manufacture's recommendations (Promega). Briefly, 100 μl of Cell Titer-Glo reagent (Promega), prepared according to manufacture's instructions, was added to each well and the plates were incubated at ambient temperature for about 10 minutes. Luciferase activity was measured to determine ATP content with a luminescence reader for 1 sec. per well.

As shown in FIG. 3, the response measured by the luciferase proliferation assay was higher compared to the ATP content method at all concentrations tested. These results indicate that proliferation or growth measured by luciferase activity provide a more robust and sensitive measurement compared to the measurement of ATP content.

To determine the correlation between cell proliferation and luciferase activity, growth curves were performed where luciferase activity and cell density and viability were determined at different times following stimulated proliferation. Briefly, 0.5×10⁴ cells/mL of cells were seeded in 30 mL growth media supplemented with 1 ng/mL of mIL-3 and 600 ug/mL of Gentincin. Cell density and viability was determined each day using a Vi-Cell Viability Analyzer (Beckman Coulter, Fullerton, California). Luciferase activity also was determined at each viability time point from a separate aliquot of 50 μL of culture media. A 50 μL aliquot of media also was used as a baseline control for each time point in the luciferase proliferation assay. Final luciferase activities were normalized to the baseline to minimize day to day variation in preparation of luciferase substrate reagents.

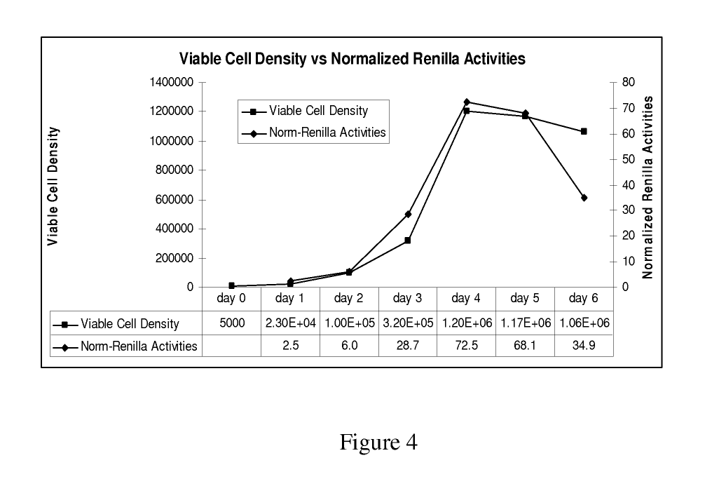

The results of this growth curve for both luciferase activity and cell density/viability are provided in FIG. 4. As shown, the 32Dcl23pRL-F11 cell growth curves for luciferase measurements closely parallel the density and viability determinations. These results indicate a direct correlation between luciferase activity and cell proliferation, growth and/or viability.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A proliferative response indicator cell, comprising a vertebrate cell having a luciferase encoding nucleic acid and a heterologous proliferation factor receptor encoding nucleic acid, wherein each of said encoding nucleic acids are operationally linked to expression elements for co-expression of a luciferase polypeptide and a heterologous proliferation factor receptor.

2. The proliferative response indicator cell of claim 1, wherein said luciferase encoding nucleic acid encodes a luciferase selected from Renilla luciferase (soft coral), Photinus pyralis luciferase (firefly) or Photobacterium fischerii luciferase (bacterial).

3. The proliferative response indicator cell of claim 1, wherein said proliferation factor receptor comprises a receptor that binds a growth factor, a cytokine or a hormone.

4. The proliferative response indicator cell of claim 1, wherein said proliferation factor receptor is selected from a receptor that binds 5637 CM, bFGF, CNTF, EGF, EPO, FTL3L, G-CSF, GDNF, GM-CSF, HGF, IFN-α, IFN-β, IFN-γ, IGF-I, IGF-II, IL-1α, IL-2, IL-3, IL-4, IL-5, IL,6, IL-7, IL-8, IL-9, IL-10, IL-11, IL-12, IL-13, IL-15, IL-18, KGF, LIF, MCP-1, M-CSF, MIP-1α, NGF, OSM, PDGF, PIXY-321, PANTES, SCF, TGF-β, TNF-α, TNF-β, TPO, insulin, growth hormone, estrogen or progesterone.

5. The proliferative response indicator cell of claim 1, wherein said vertebrate cell comprises a mammalian cell.

6. The proliferative response indicator cell of claim 5, wherein said mammalian cell comprises a human or mouse cell.

7. The proliferative response indicator cell of claim 5, wherein said vertebrate cell is selected from a group consisting of 32D, an IL-3 dependent 32D cell, AML-193, ELF-153, F-36P, GF-D8, GM/SO, HU-3, M-07e, MB-02, MHH-203, M-MOK, MUTZ-2, MUTZ-3, OCI/AML1, OCI/AML5, OCI/ML6, SKNO-1, TF-1, UCSD/AML1, UT-7, 293, NIH3T3, Hela, CHO, CV-1, PC-3, Jurkat and NFS60, or a genetically modified cell thereof expressing said proliferation factor receptor.

8. The proliferative response indicator cell of claim 1, wherein said expression element operationally linked to said luciferase encoding nucleic acid comprises a promoter non-responsive to a proliferation factor bound by said proliferation factor receptor.

9. The proliferative response indicator cell of claim 8, wherein said promoter comprises a constitutive promoter.

10. A method of determining a cell proliferative response to a proliferation factor, comprising:
(a) contacting a vertebrate cell expressing luciferase and a proliferation factor receptor with a proliferation factor for sufficient time for said proliferation factor to bind to said proliferation factor receptor;
(b) culturing said contacted cell expressing luciferase for at least one generation, and
(c) measuring the amount of light emission, wherein said luciferase expression is driven from a promoter non-responsive to said proliferation factor and said light emission directly correlates with proliferation factor-mediated cell proliferation.

11. The method of claim 10, wherein said proliferation factor comprises a growth factor, a cytokine or a hormone.

12. The method of claim 10, wherein said proliferation factor is selected from 5637 CM, bFGF, CNTF, EGF, EPO, FTL3L, G-CSF, GDNF, GM-CSF, HGF, IFN-α, IFN-β, IFN-γ, IGF-I, IGF-II, IL-1α, IL-2, IL-3, IL-4, IL-5, IL,6, IL-7, IL-8, IL-9, IL-10, IL-11, IL-12, IL-13, IL-15, IL-18, KGF, LIF, MCP-1, M-CSF, MIP-1α, NGF, OSM, PDGF, PIXY-321, PANTES, SCF, TGF-β, TNF-α, TNF-β, TPO, insulin, growth hormone, estrogen or progesterone.

13. The method of claim 10, wherein said proliferation factor comprises an agonist to said proliferation factor receptor.

14. The method of claim 10, further comprising determining the effect of an inhibitor of said proliferation factor, said method comprising the step of contacting said vertebrate cell expressing luciferase and a proliferation factor receptor with a proliferation factor in the presence of a sample suspected of containing an inhibitor of said proliferation factor, wherein a decrease of cell proliferation in the presence of said sample indicates that said sample includes an inhibitor of said proliferation factor.

15. The method of claim 14, wherein said inhibitor of said proliferation factor comprises a neutralizing antibody, or binding fragment thereof, to said proliferation factor.

16. The method of claim 14, wherein said inhibitor of said proliferation factor comprises a competitive or non-competitive inhibitor said proliferation factor receptor.

17. The method of claim 14, wherein said inhibitor of said proliferation factor comprises an antagonist to said proliferation factor receptor.

18. The method of claim 10, wherein said luciferase is selected from Renilla luciferase (soft coral), Photinus pyralis luciferase (firefly) or Photobacterium fischerii luciferase (bacterial).

19. The method of claim 10, wherein said vertebrate cell comprises a mammalian cell.

20. The method of 19, wherein said mammalian cell comprises a human or mouse cell.

21. The method of claim 19, wherein said vertebrate cell is selected from a group consisting of 32D, an IL-3 dependent 32D cell, AML-193, ELF-153, F-36P, GF-D8, GM/SO, HU-3, M-07e, MB-02, MHH-203, M-MOK, MUTZ-2, MUTZ-3, OCI/AML1, OCI/AML5, OCI/ML6, SKNO-1, TF-1, UCSD/AML1, UT-7, 293, NIH3T3, Hela, CHO, CV-1, PC-3, Jurkat and NFS60, or a genetically modified cell thereof expressing said proliferation factor receptor.

22. The method of claim 21, wherein said genetically modified cell expresses a receptor for thrombopoietin (TPO).

23. The method of claim 10, wherein said promoter non-responsive to said proliferation factor comprises a constitutive promoter.

24. The method of claim 23, wherein said constitutive promoter is selected from the group of promoters consisting of CMV, thymidine kinase (tk), SV40 and phosphoglycerate kinase (PGK).

25. The method of claim 10, wherein said direct correlation with said proliferation factor-mediated cell proliferation further comprises an indicator of cell health or viability.

26. The method of claim 10 or 25, wherein said direct correlation with said proliferation factor mediated cell proliferation comprises a signal substantially free of proliferation factor-mediated transcriptional effects.

27. A diagnostic system, comprising a plurality of different vertebrate cell lines each encoding a luciferase gene and a different proliferation factor receptor, said luciferase gene being operationally linked to a promoter non-responsive to a proliferation factor bound by said proliferation factor receptor, wherein light emission from each of said different cell lines being characterized as directly correlating with proliferation factor-mediated cell proliferation.

28. The diagnostic system of claim 27, wherein said plurality of different vertebrate cell lines comprises three or more different cell lines.

29. The diagnostic system of claim 27, wherein said plurality of different vertebrate cell lines comprises 10 or more different cell lines.

30. The diagnostic system of claim 27, further comprising a proliferation factor corresponding to at least one of said different proliferation factor receptors.

31. The diagnostic system of claim 27, further comprising one or more ancillary reagents.

32. The diagnostic system of claim 27, wherein said proliferation factor receptor comprises a receptor for a growth factor, a cytokine or a hormone.

33. The diagnostic system of claim 27, wherein said proliferation factor is selected from 5637 CM, bFGF, CNTF, EGF, EPO, FTL3L, G-CSF, GDNF, GM-CSF, HGF, IFN-α, IFN-β, IFN-γ, IGF-I, IGF-II, IL-1α, IL-2, IL-3, IL-4, IL-5, IL,6, IL-7, IL-8, IL-9, IL-10, IL-11, IL-12, IL-13, IL-15, IL-18, KGF, LIF, MCP-1, M-CSF, MIP-1α, NGF, OSM, PDGF, PIXY-321, PANTES, SCF, TGF-β, TNF-α, TNF-β, TPO, insulin, growth hormone, estrogen or progesterone.

34. The diagnostic system of claim 30, wherein said proliferation factor comprises an agonist to said proliferation factor receptor.

35. The diagnostic system of claim 31, wherein said ancillary reagent comprises inhibitor of said proliferation factor

36. The diagnostic system of claim 35, wherein said inhibitor of said proliferation factor is selected from a neutralizing antibody, or binding fragment thereof, to a proliferation factor bound by said proliferation factor receptor, a competitive inhibitor, a non-competitive inhibitor or an antagonist to said proliferation factor receptor.

37. The diagnostic system of claim 27, wherein said luciferase gene is selected from Renilla luciferase (soft coral), Photinus pyralis luciferase (firefly) or Photobacterium fischerii luciferase (bacterial).

38. The diagnostic system of claim 27, wherein said vertebrate cell comprises a mammalian cell.

39. The diagnostic system of 38, wherein said mammalian cell comprises a human or mouse cell.

40. The diagnostic system of claim 39, wherein said vertebrate cell is selected from a group consisting of 32D, an IL-3 dependent 32D cell, AML-193, ELF-153, F-36P, GF-D8, GM/SO, HU-3, M-07e, MB-02, MHH-203, M-MOK, MUTZ-2, MUTZ-3, OCI/AML1, OCI/AML5, OCI/ML6, SKNO-1, TF-1, UCSD/AML1, UT-7, 293, NIH3T3, Hela, CHO, CV-1, PC-3, Jurkat and NFS60, or a genetically modified cell thereof expressing said proliferation factor receptor.

41. The diagnostic system of claim 30, wherein said genetically modified cell expresses a receptor for thrombopoietin (TPO).

42. The diagnostic system of claim 27, wherein said promoter non-responsive to said proliferation factor comprises a constitutive promoter.

43. The diagnostic system of 42, wherein said constitutive promoter is selected from the group of promoters consisting of CMV, thymidine kinase (tk), SV40 and phosphoglycerate kinase (PGK).

44. The diagnostic system of claim 27, wherein said direct correlation with said proliferation factor-mediated cell proliferation further comprises an indicator of cell health or viability.

45. The diagnostic system of claim 27 or 44, wherein said direct correlation with said proliferation factor mediated cell proliferation comprises a signal substantially free of proliferation factor-mediated transcriptional effects.
